(19) United States
(12) Patent Application Publication
(10) Pub. No.: US 2002/0072737 A1
(43) Pub. Date: Jun. 13, 2002

(54) System and method for placing a medical electrical lead

(75) Inventors: Elisabeth Lacy Belden, Maple Grove, MN (US); Roger Dahl, Andover, MN (US); John Louis Sommer, Coon Rapids, MN (US); Thomas M. Soukup, Plymouth, MN (US); Steve Sundquist, Minnetonka, MN (US); Arnold Thornton, Roseville, MN (US); Duane Zytkovicz, Ham Lake, MN (US)

Correspondence Address: Beth L. McMahon, Medtronic, Inc., Ms 300, 7000 Central Avenue NE, Minneapolis, MN, 55432, US

(73) Assignee: Medtronic, Inc.

(21) Appl. No.: 09/836,017

(22) Filed: Apr. 17, 2001

Related U.S. Application Data
(60) Provisional application No. 60/254,102, filed on Dec. 8, 2000.

(51) Int. Cl. A61B 18/04

(52) U.S. Cl. 606/34; 606/129

(57) ABSTRACT

A system and method for deploying a lead in a cardiac chamber, a cardiac vein, or a coronary artery of a patient is disclosed. The system includes a delivery device such as a guidewire having an electrode retention member at the guidewire distal end to engage an electrode assembly. The guidewire is adapted to be inserted into the inner lumen of an introducer sheath so that the electrode retention member extends beyond the distal tip of the introducer. The electrode assembly is then coupled to the electrode retention member of the guidewire. The introducer includes means at the distal end adapted to engage the proximal end of the electrode assembly that is mounted on the guidewire. This allows the introducer to push the electrode assembly and the guidewire through the vasculature to a predetermined point of implant. The introducer may then be utilized to dislodge the electrode assembly from the guidewire at the predetermined implant site before the guidewire and introducer are withdrawn. In one embodiment, the guidewire includes a steerable distal tip to allow the assembly to be readily maneuvered to the implant site. The electrode retention member may be rotatably mounted on the guidewire so that the guidewire steerable distal tip is free to rotate through the curves of a patient's vascular system without requiring rotation of the electrode assembly. According to one aspect of the invention, the guidewire and introducer may be readily coupled to a previously-implanted electrode assembly so the electrode may be re-located at a second implant site.

### RELATED APPLICATIONS

This Application claims priority to provisionally-filed U.S. patent application Ser. No. 60/254,102 filed Dec. 8, 2000 entitled “System and Method for Placing a Medical Electrical Lead”, which is incorporated herein by reference in its entirety.

### FIELD OF THE INVENTION

The present invention relates generally to a system and method for placing one or more implantable cardiac leads within a coronary artery or cardiac vein; and more particularly, relates to a system including a guidewire having a distal tip member for engaging an electrode assembly during electrode placement and re-positioning procedures, and an introducer sheath having a distal section capable of dislodging the electrode assembly from the guidewire.

### BACKGROUND OF THE INVENTION

Implantable medical electrical stimulation and/or sensing leads are well known in the fields of cardiac stimulation and monitoring, including cardiac pacing and cardioversion/defibrillation. In the field of cardiac stimulation and monitoring, endocardial leads are placed through a transvenous route to locate one or more sensing and/or stimulation electrodes along or at the distal end of the lead in a desired location within a heart chamber or interconnecting vasculature. In order to achieve reliable sensing of the cardiac electrogram and/or to apply stimulation that effectively paces or cardioverts the heart chamber, it is necessary to accurately position the electrode surface against the endocardium, pericardium, or within the myocardium at the desired site and fix it during an acute post-operative phase until fibrous tissue growth occurs.

The pacemaker or defibrillator implantable pulse generator (IPG) or the monitor is typically coupled to the heart through one or more of such endocardial leads. The proximal end of such a lead is typically formed with a connector which connects to a terminal of the IPG or monitor. The lead body typically comprises one or more insulated conductive wire surrounded by an insulating outer sleeve. Each conductive wire couples a proximal lead connector element with a distal stimulation and/or sensing electrode. An endocardial cardiac lead having a single stimulation and/or sensing electrode at the lead distal end and a single conductive wire is referred to as a unipolar lead. An endocardial cardiac lead having two or more stimulation and/or sensing electrodes at the lead distal end and two or more conductive wires is referred to as a bipolar lead or a multi-polar lead, respectively.

In order to implant an endocardial lead within a heart chamber, a transvenous approach is utilized wherein the lead is inserted into and passed through the subclavian, jugular, or cephalic vein and through the superior vena cava into the right atrium or ventricle. An active or passive fixation mechanism is incorporated into the distal end of the endocardial lead and deployed to maintain the distal end electrode in contact with the endocardium position.

More recently, endocardial pacing and cardioversion/defibrillation leads have been developed that are adapted to be advanced into the coronary sinus and coronary veins branching therefrom in order to locate the distal electrode(s) adjacent to the left ventricle or the left atrium. The distal end of such coronary sinus leads is advanced through the superior vena cava, the right atrium, the valve of the coronary sinus, the coronary sinus, and may further be advanced into a coronary vein communicating with the coronary sinus, such as the great vein. Typically, coronary sinus leads do not employ any fixation mechanism and instead rely on the close confinement within these vessels to maintain each electrode at a desired site.

Routing an endocardial lead along a desired path to implant the electrode or electrodes in a desired implantation site, either in a chamber of the heart or in the selected cardiac vein or coronary artery, can be difficult. This is particularly true for steering leads through the coronary sinus and into a branching vein on the left myocardium. Anomalies in the vascular anatomy and the number of branch veins associated with the anatomy make locating the desired path challenging.

Several common approaches have been developed to place electrodes within the left side of the heart. According to one approach, a guide catheter is steered into the desired location in the vasculature. A lead is then fed through the inner lumen of the catheter such that the lead electrode(s) are positioned at predetermined locations. The guide catheter may then be withdrawn. This type of approach is described in commonly assigned U.S. Pat. Nos. 6,006,137, 5,246,014, and 5,851,226 incorporated herein by reference. The described systems employ highly flexible, catheters surrounding the lead body. One difficulty with systems of this nature is that the lead body may not have the necessary stiffness properties to be pushable through the catheter lumen. This is particularly true when the catheter is positioned within the torturous curves of a patient's vasculature system. The problem is exaggerated when very small leads having a diameter of 4 French or less are employed for use in the coronary sinus or associated vasculature.

Another approach to lead placement involves the use of a guide wire that is steered into a desired location within the vasculature. The lead body is then tracked over the wire and the wire is withdrawn. According to this design, the guide wire passes through an inner lumen of the lead for an entire length of the lead. This results in a significant amount of friction that can make lead placement difficult. Additionally, since the lead must include an inner lumen for the guide wire, the size of the lead is at least somewhat dictated by the size of the guide wire. Moreover, to accomplish lead placement in this manner, the lead must again be stiff enough to allow it to be advanced over the guide wire through the tortuous curves of the vasculature.

Yet another approach is described in commonly-assigned U.S. Pat. No. 5,902,331 to Bonner et al. The disclosed system includes a pusher mechanism that is adapted to slidably engage a guidewire that has previously been placed at a desired implant site. The pusher mechanism couples to a lead body to allow the pusher to guide the lead over the guidewire to the desired implant site. The lead body may then be released from the pusher, and the pusher and guidewire are withdrawn from the body. The disclosed system is not easily used to re-position the electrode assembly once the electrode resides within the vasculature, however.

What is needed is another method for placing leads within the vasculature including the coronary sinus that does not require a lead having an inner lumen, and that further does not require use of a lead having a body stiffness sufficient to allow the lead to be pushed over a guidewire or through the inner lumen of a catheter. The system should also be capable of deploying electrodes over previously-placed leads. Ideally, the system would also allow for the re-positioning of electrodes within the vasculature. This may be necessary to ensure capture, for example, or to provide additional electrodes such as defibrillation electrodes not provided at the time of initial implant.

### SUMMARY OF THE INVENTION

The present invention provides a system and method for deploying a lead in a cardiac chamber or in the cardiac veins or coronary arteries of a body. The system includes a delivery device such as a guidewire having an electrode retention member to engage a hollow electrode assembly. The guidewire is adapted to be inserted into the inner lumen of an introducer sheath so that the electrode retention member extends beyond the distal tip of the introducer. The electrode assembly is then coupled to the electrode retention member of the guidewire. The entire assembly may then be navigated to a desired site of implant.

According to one aspect of the invention, the introducer of the current invention may include means at the distal end adapted to engage the proximal end of the electrode assembly when the electrode assembly is mounted on the guidewire. This allows the introducer to push the electrode assembly and the guidewire through the vascular system to a predetermined point of implant.

In one embodiment of the invention, the guidewire includes a distal tip that extends distally beyond the electrode assembly, and which may be manually shaped prior to advancing the assembly through the vasculature. Alternatively, the guidewire may include at least one tension wire coupled to the distal tip and extending to the proximal end of the guidewire to deflect the distal tip as the assembly is maneuvered through the vasculature. The introducer sheath transmits the push force to manipulate the assembly into place, thus making it unnecessary to utilize a lead body having a substantial degree of stiffness.

When the implant site has been located, the introducer sheath is advanced distally, or alternatively, the guidewire is pulled in a proximal direction, to force the electrode assembly from the guidewire. Then the introducer sheath and guidewire may be withdrawn from the vasculature, leaving the electrode in position.

In one embodiment of the invention, the introducer lumen is large enough to accommodate both the guidewire and the lead of the electrode assembly. In this embodiment, the introducer must be retracted over the lead body after the electrode is deployed. To facilitate this, a low-profile connector may be used, or a splittable embodiment of introducer may be employed. In another embodiment of the introducer, the lead for the electrode assembly is maintained at a position adjacent an external surface of the introducer. This lead position is maintained using a coupling mechanism or device such as grooved member located at a proximal end of the introducer. The grooved member is adapted to engage a proximal end of the lead during the implant procedure. This grooved member may be provided at a location on the introducer that remains external to a body during the implant procedure so that the lead may be readily de-coupled from the introducer after the electrode is located at the implant site. Then the introducer and guidewire may be removed from the body, leaving the lead and electrode in place.

In one embodiment, the introducer comprises an outer tubular member and an inner metal coil. The metal coil may be coated with a thin layer of flexible or polymeric material and the distal end is terminated with a solid, rigid, and radiopaque tube. The metal coil prevents kinking as the introducer is advanced through the vasculature, and farther reduces friction when a device such as the guidewire is advanced and retracted through the introducer inner lumen. The radiopaque tube provided at the distal end of introducer allows the position of the electrode assembly, which is, by its nature, radiopaque, to be tracked with respect to the position of the distal end of the introducer by fluoroscopy as the lead is being implanted within the vascular system.

If a splittable version of an introducer is employed in the manner discussed above, the introducer does not include a metal coil, but instead includes a tubular body formed of biocompatible polymer. The introducer of this embodiment may include a weakened longitudinal seam used for splitting the introducer. Additionally, a radiopaque tube is formed as a “C” ring to facilitate splitting of the introducer at the distal tip. According to one aspect of the invention, a splittable introducer may be formed of a corrugated or pleated tubular member that resists kinking and decreases friction when a device is inserted or retracted from the inner lumen of the introducer.

As discussed above, the guidewire includes an electrode retention member adapted to couple to the electrode assembly during the implant procedure. In one embodiment, this retention mechanism is an enlarged plug that forms a press fit with an inner lumen of the electrode assembly. This plug may include a lumen adapted to fit within a grooved section of the guidewire, allowing the plug to rotate freely around the guidewire. This allows the guidewire to be steered independently of the plug and electrode assembly as the introducer, guidewire, and electrode assembly are guided to the implant site. The press fit is selected such that the electrode assembly will not slide off the guidewire during navigation to the implant site. However, the electrode assembly can easily be dislodged from the electrode retention member by sliding the introducer forward to apply force to the electrode assembly as tension is applied to the proximal end of the guidewire.

In another embodiment, the electrode retention member comprises the distal tip of the guidewire that is formed in a serpentine curve having an outer diameter sized to form a press fit with the inner diameter of the electrode assembly. In yet a third embodiment, the retention member is a tubular core having flexible bristles extending radially to engage the inner surface of the electrode assembly.

In yet another embodiment of the guidewire, the electrode retention member is an inflatable member such as a balloon. The guidewire includes an injection port at the proximal end to allow for inflation of the inflation member via an internal lumen of the guidewire. The inflation member is inflated to engage an electrode assembly, and is later deflated when the electrode assembly is to be deployed.

According to one method of using the introducer and electrode assembly of the current invention, the electrode assembly may be threaded over the proximal end of a lead that carries one or more electrodes already placed within a patient's vascular system. Next, the introducer of the current invention is also placed over the proximal end of the lead so that the distal end of the introducer engages the electrode assembly. A push force applied to the proximal end of the introducer may then be used to advance the electrode assembly along the previously-placed lead body to a desired implant site along the lead body before the introducer is withdrawn. This method may be used to place a defibrillation coil at a predetermined site along the body of a previously-placed lead that carries one or more pacing or defibrillation electrodes. For example, it may be desirable to place an additional defibrillation electrode at a location, through which a previously-placed lead body passes, in order to reduce the defibrillation threshold of the system. Alternatively, this method may be employed if a unipolar pacing electrode configuration is to be converted to a bipolar configuration, or if one or more additional sensing electrodes are desired.

According to another method of use, the current inventive system may be employed to re-locate a previously-placed electrode assembly. This may be desirable, for example, in the case wherein a defibrillation electrode was placed at a location having a defibrillation threshold that is later determined to be too high. To accomplish the necessary electrode re-location, the guidewire is advanced through the inner lumen of the introducer so that the guidewire distal tip extends beyond the distal end of the introducer. The guidewire and introducer are then navigated through the vasculature to the original site of implant. If desired, the distal tip of the guidewire may be retracted to allow a tapered edge of the introducer to mate with the electrode assembly, thereby centering the inner lumen of the introducer with the inner lumen of the electrode assembly. The distal tip of the guidewire may be advanced as pressure is maintained on the proximal end of the electrode lead, thereby allowing the electrode retention member of the guidewire to mate with the inner lumen of the electrode assembly. The guidewire, introducer, and electrode assembly may then be navigated to a second predetermined implant site, where the electrode assembly is deployed by advancing the introducer distally to push the electrode assembly off the electrode retention member. The guidewire and introducer may then be withdrawn.

Other aspects of the current invention will become apparent to those skilled in the art from the following detailed description, and the accompanying figures.

### BRIEF DESCRIPTION OF THE DRAWINGS

FIG. 1 is a cutaway view illustrating one embodiment of the introducer of the current invention.

FIG. 2A is a side view of another embodiment of a splittable introducer that may be used in accordance with the current invention.

FIG. 2B is a cross-sectional view of the introducer of FIG. 2A at line 2B-2B including the “C” ring construction of the tubular member.

FIG. 3A is a cutaway side view of an exemplary guidewire construction that may be used in conjunction with the current invention.

FIG. 3B is a cross-sectional view of the guidewire of FIG. 3A at line 3B-3B.

FIG. 4 is an exploded side view of an electrode assembly that is adapted for use with the guidewire of FIG. 3.

FIG. 5 is a cross-sectional end view of an electrode illustrating the manner in which the lead of FIG. 4 is coupled to coil.

FIG. 6 is a cutaway side view showing one manner in which the guidewire may be used in conjunction with the introducer of the current invention to deploy the electrode.

FIG. 7 is a cutaway side view of the introducer and guidewire, and illustrates another embodiment of the electrode assembly.

FIG. 8 is a cross-sectional view of the introducer at line 8-8 of FIG. 7, and illustrates the lead body retained in a grooved member.

FIG. 9 is a side plan view showing an alternative embodiment of a guidewire that may be used in conjunction with the current invention.

FIG. 10 is a side plan view showing an alternative embodiment of a guidewire that includes a shorter distal tip region 86 than is provided by previously-discussed guidewire designs.

FIG. 11A is a perspective view of yet another embodiment of the guidewire including a brush member having soft, deformable bristles extending radially from the guidewire body.

FIG. 11B is yet another embodiment of the current invention in which the retention device is an inflatable member such as a balloon provided on the body of the guidewire.

FIG. 12 is a cutaway side view illustrating another embodiment of the introducer.

FIG. 13 is a cutaway side view illustrating a previously implanted lead body being used to implant additional electrodes.

FIG. 14 illustrates the manner in which the current inventive system may be employed to reposition an electrode from a first implant site to another location.

FIG. 15 is a side cutaway view illustrating the manner in which an embodiment of the introducer may also be employed to deploy an electrode assembly that does not have a lumen for the passage of a guidewire.

FIG. 16 is a cross-sectional view of the introducer at line 16-16 of FIG. 15 illustrating a pull-wire housed within a lumen in the side wall of the introducer.

### DETAILED DESCRIPTIONS OF THE DRAWINGS

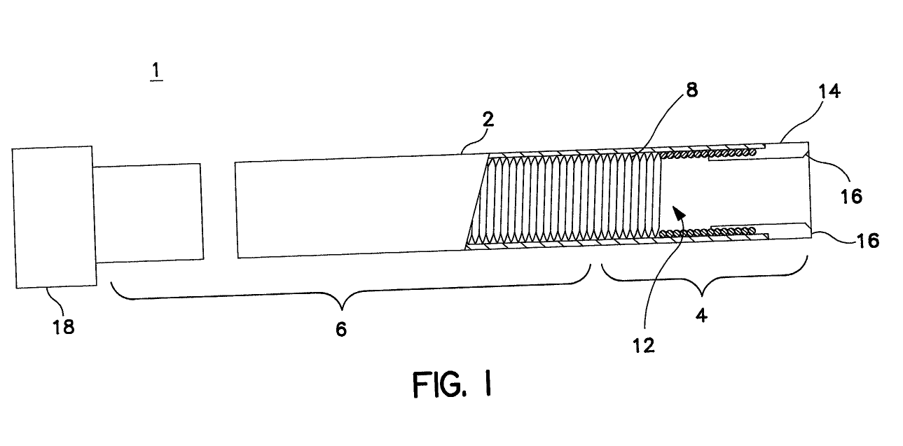

FIG. 1 is a cutaway view illustrating one embodiment of introducer 1 of the current invention. The introducer may include a flexible polymeric layer 2, which may have a structure corresponding to the body of a conventional guide catheter or introducer such as that corresponding to the SHERPA® guide catheter commercially-available from the Medtronic Corporation. Other exemplary catheter structures are disclosed in U.S. Pat. No. 5,755,704 issued to Lunn, U.S. Pat. No. 5,545,149 issued to Brin, et al., and U.S. Pat. No 5,811,043 issued to Horrigan, et al., all incorporated herein by reference in their respective entireties. Polymeric layer 2 may be formed of any type of biocompatible silicon, polyurethane, polyethylenes, polyesters, polyether block amides, polyamides, polytetrafluoroethylenes and the like. This layer may be reinforced by polymeric or metallic braids or wires. Alternatively, this layer may take the form of an un-reinforced tube of any of the materials referred to above.

In one embodiment, polymeric layer 2 may have several different stiffness ranges. For example, a distal region 4, which may be between 1 and 8 cm in length, may be formed of a material that is less stiff than a proximal region 6. If desired, both the proximal and distal regions could each include multiple stiffness regions such that the introducer shaft becomes increasingly more flexible closer to the distal tip. This decreases the risk of perforation or vascular damage during use.

The introducer may include a conductive coil 8 of conventional design. The coil allows the introducer 1 to resist kinking as it is inserted into a patient's vasculature. This coil also reduces drag when an object such as a stylet is introduced into the inner lumen of the device. In another embodiment, the coil may be replaced by an inner sheath formed of a lubricious material such as a silicone or a polymeric material including polyN-vinylpyrrolidone, hydrophilic polyurethanes, or Teflon.

In the embodiment shown in FIG. 1, metal coil 8 defines an inner lumen 12. In one embodiment of the invention, the diameter of the inner lumen 12 ranges from approximately 0.020 inches to 0.090 inches. The wall thickness of the introducer including the coil may range from approximately 0.008 inches to 0.015.

The distal tip of introducer 1 may include a radiopaque tubular member 14 that both terminates coil 8, and that is designed to interface with an electrode assembly (not shown in FIG. 1). In one embodiment, tubular member is approximately 0.8 inches long, but other lengths ranging from about 0.6 to 0.10 inches may be used depending on the configuration of the electrode assembly that is to interface to the tubular member. The inner diameter of the tubular member 14 is at least approximately 0.010 inches smaller than the outer diameter of any electrode assembly that is to be used with the introducer, as will be discussed further below. The distal tip of tubular member 14 defines a nesting taper 16 to aid in the alignment of the electrode assembly. It should be noted that the distal end of the introducer may also be formed by fusing several turns of coil 8.

Introducer 1 may further include a handpiece 18 which may be formed of a molded plastic which includes a proximal port that is in fluid communication with lumen 12. Handpiece 18 may be coupled to the introducer sheath via a toughy-borst seal (not shown) or some other type of catheter connector known in the art. This connector closes around any device such as a guidewire that is advanced through the proximal port into lumen 12 to constrain the relative motion of the introducer and the device.

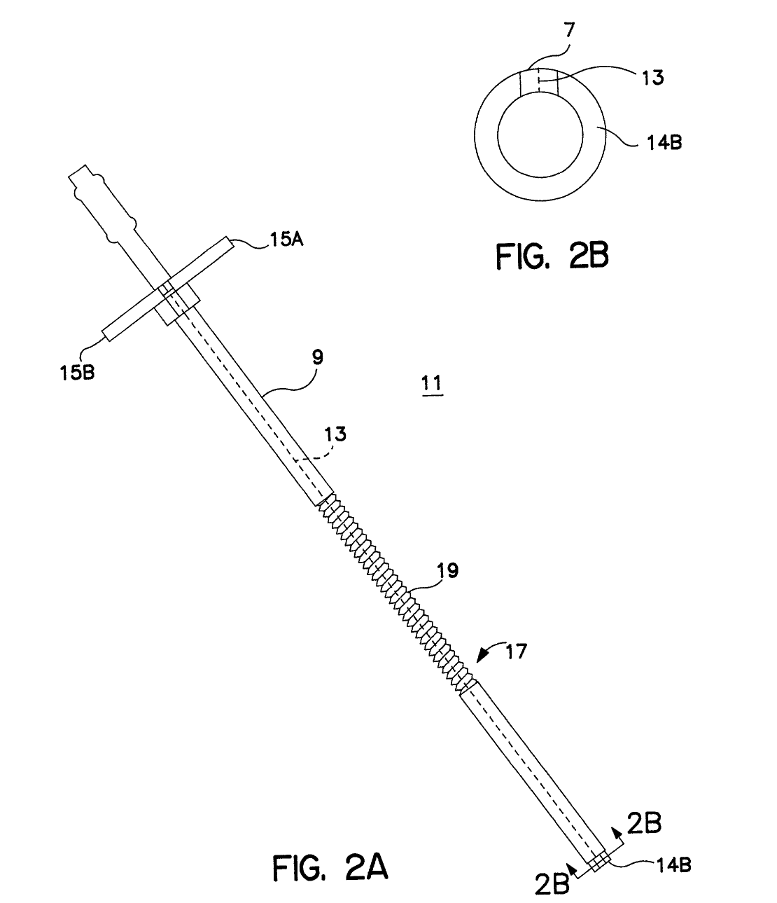

FIG. 2A is a side view of another embodiment of a splittable introducer that may be used in accordance with the current invention. This embodiment of the introducer, which is disclosed in commonly-assigned U.S. Pat. No. 4,409,469 to Schaerf entitled “Introducer System Having Kink Resistant Splittable Sheath” incorporated herein in its entirety, is splittable. Specifically, introducer 11 may be longitudinally split along line 13 by grasping tabs 15A and 15B as it is being withdrawn from the introduction site. In the preferred embodiment, line 13 comprises a scoring within wall of introducer 11 as is known in the art. Various other equivalent means may also be used to accomplish splitting sheath 11 along line 13, such as providing a linear region of the wall that is weakened, as shown in Vegoe et al U.S. Pat. No. 5,180,372, incorporated herein by reference. The weakened wall section may consist of material having the physical property of molecular orientation whereby a tear in the material runs readily only in a longitudinal direction along the length of introducer 11, as is known in the art. Alternatively, a sheath slitter or the like may be used to split the introducer.

To facilitate splitting, introducer 11 does not include coil 8. The walls 9 of the introducer are formed of a biocompatible plastic, such as polytetrafluoroethylene. Additionally, flouroscopic tubular member 14 (FIG. 1) is configured in this embodiment as a “C” ring 14B having an opening to allow passage of a slitting tool. To reduce friction with inner walls of the introducer so that devices such as the guidewire may be more readily advanced and retracted within the inner lumen, a pleated or corrugated wall construction may be used. The pleated wall construction further prevents kinking of the device as the introducer is advanced within the coronary vasculature. This contruction is shown in FIG. 2, which includes one or more kink resistant sections 17 having a series of pleats 19. The entire introducer may be formed of the kink resistant configuration, if desired. Additionally, the inner walls may further include a lubricious coating such as Teflon to further aid in reducing friction.

FIG. 2B is a cross-sectional view of introducer at line 2B-2B of FIG. 2A, including “C” ring 14B. A portion 7 of the distal end of introducer 11 may be formed of the material included in the weakened portion of the wall, as shown by dashed line 13. This allows the introducer to be easily split. Alternatively, in a slittable version of introducer, some or all of portion 7 is formed of the same material included in the walls 9 of the introducer such as polytetrafluoroethylene.

In the preferred embodiment of the invention, the introducer is adapted to receive a guidewire. The body of the guidewire may be of any conventional design such as that described in commonly-assigned U.S. Pat. No. 4,815,478 to Buchbinder et al., incorporated herein by reference in its entirety. Suitable alternative guidewire systems are described in U.S. Pat. Nos. 5,095,915, 4,545,390, or 5,746,710, all incorporated by reference herein in their entirety.

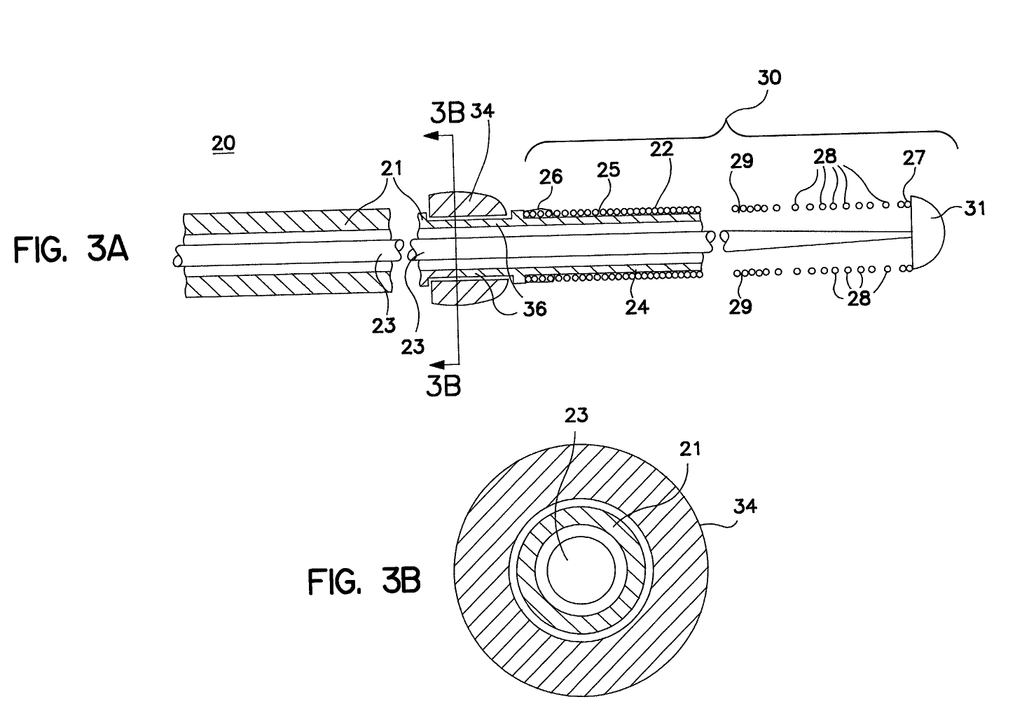

FIG. 3A is a cutaway side view of an exemplary guidewire construction that may be use in conjunction with the current invention. In one embodiment, guidewire 20 comprises tubing 21 and spring coil 22 disposed around the tubing at the guidewire distal tip. The tubing 21 may be formed of any biocompatible material that exhibits the required physical properties to allow the guidewire to be advanced through tortuous or curved passageways or ducts of the body. In such embodiments, the tubing 21 may be formed of stainless steel hypodermic tubing, or alternatively, of other types of flexible biocompatible plastic medical tubing. In one embodiment, the diameter of guidewire may range from approximately 0.014 to 0.020 inches, although larger or smaller dimensions may be selected depending on intended use.

The distal portion 24 of tubing 21 may be recessed such that the proximal portion 25 of spring coil 22 over the tubing. This provides an atraumatic distal portion 30. The proximal portion 25 of spring coil 22 may be affixed to tubing 21, preferably by welding, brazing, soldering, or by using a medical grade adhesive at adhesion point 26. The coils of spring coil 22 proximal to distal end 27 may comprise stretched coils 28, which extend from distal end 27 to adhesion point 29 to further reduce the tip stiffness. The distal end 27 of stretched coils 28 are shown engaged by cap or tip 31. In one embodiment, the distal portion 30 is between 0.7 inches to 1.6 inches in length and may range in stiffness depending on the desired use.

In yet another embodiment, the guidewire does not include the coil at the distal portion 30 such that tubing 21 extends the entire length of the guidewire in a uniform thickness.

Guidewire 20 may include a deflection wire 23 that extends the length of the guidewire and that is affixed to cap 31. The deflection wire 23 provides a means to deflect the distal tip section when tension is applied to the proximal end of the deflection wire. By attaching the deflection wire to a position of cap 31 that is off-center from the center axis of the guidewire, a preferred bending direction is provided for the guidewire. If guidewire 20 does not include a deflection wire 23, steering may be accomplished by manually shaping distal tip portion 30 prior to guidewire deployment.

Guidewire 20 as shown in FIG. 3A further includes an enlarged diameter plug 34 provided proximal to the distal tip portion 30. This plug may be formed of any biocompatible material including a biocompatible polymer or a biocompatible metal material. The plug is a cylindrical structure having an inner lumen that is adapted to ride within a grooved portion 36 of tubing 21 proximal to the distal portion 30. Grooved portion 36 maintains plug 34 in a stationary longitudinal position along the body of guidewire 20. The lumen of plug 34 is large enough to allow the guidewire to rotate independently of the plug. This freedom of movement allows the guidewire to be readily steered through the cardiac vasculature without being restricted by an electrode that is retained on plug. This is discussed further below.

FIG. 3B is a cross-sectional view of guidewire 20 at line 3B-3B of FIG. 3A. Plug 34 is shown surrounding grooved portion 36 of tubing 21. In this embodiment, deflection wire 23 is included in the lumen of guidewire 20, although this is not a requirement.

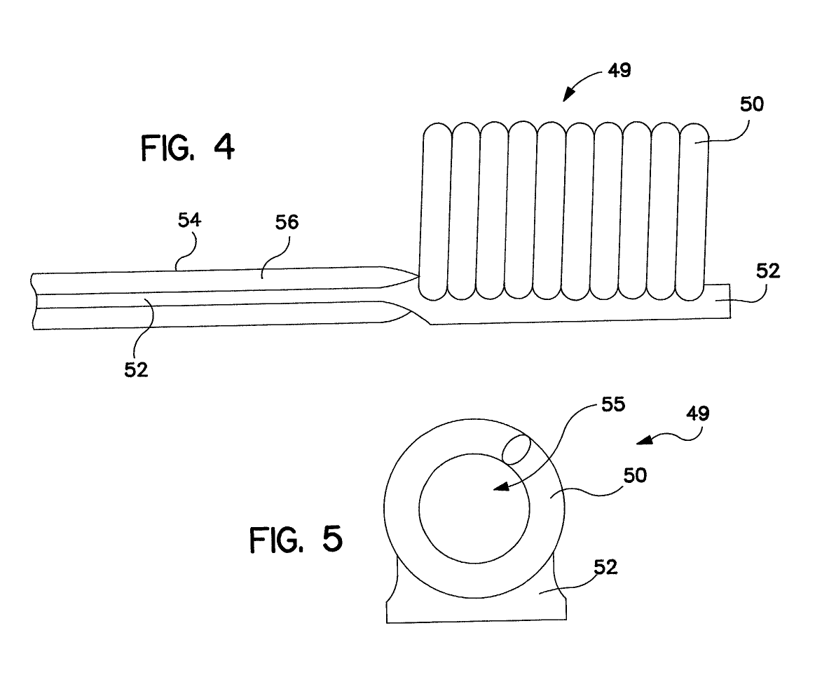

FIG. 4 is an exploded side view of an electrode assembly 49 that is adapted for use with the guidewire 20 of FIGS. 3A and 3B. The annular electrode assembly 50 of this embodiment is a coil formed of one or more conductors. This electrode assembly could be used for pacing, cardioversion/defibrillation, or the delivery of other types of electrical stimulation. If desired, multiple electrodes separated by an insulation layer may be included in electrode assembly 50. The inner diameter of coil is sized to be just slightly larger than the outer diameter of plug 34 to couple to plug using a press fit as will be discussed further below. In one embodiment of the invention, the outer diameter of the electrode assembly 49 may range from approximately 0.040 to 0.100 inches, with an inner diameter that ranges from approximately 0.030 to 0.090 inches. The length of electrode assembly 50 in this embodiment may range from approximately 0.150 to 2.4 inches.

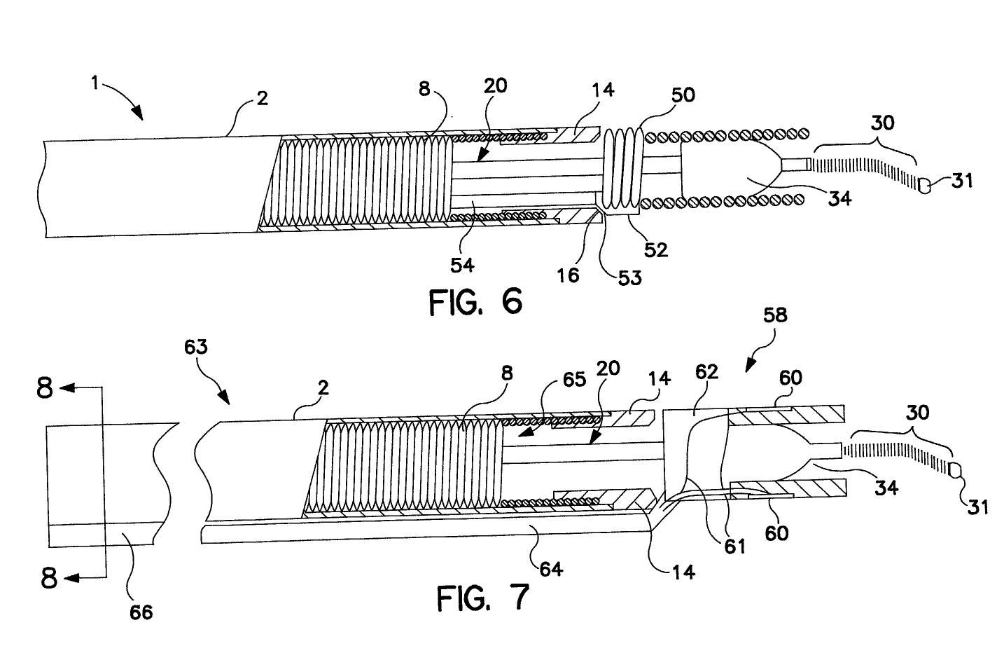

The electrode coil may be coupled to a conductor 52 using a crimping, soldering, welding, or brazing process. FIG. 4 shows coil coupled to conductor 52 along the entire longitudinal length, although this is not a requirement. In one embodiment, conductor 52 may be covered with an insulative material. Conductor may have a notched proximal edge 53 that can be aligned with nesting taper 16 of tubular member 14 as shown further in FIG. 6. This aids in centering the electrode assembly 49 at the distal end of introducer 1.

The conductor extends to the proximal end of lead 54, and may be coupled to an implantable pulse generator as is known in the art. Lead 54 may include one or more additional electrodes along the lead body, each coupled to a different respective conductor for multipolar applications. The lead may include an insulated outer layer 56 that may be biocompatible silicone, polyurethane, or another biocompatible material known in the art for this purpose.

FIG. 5 is a cross-sectional end view of the electrode assembly 49. This view shows the manner in which conductor 52 is coupled to one edge of coil 50 such that the inner lumen 55 is unobstructed and can receive guidewire 20, as is discussed below.

FIG. 6 is a cutaway side view showing one manner in which guidewire 20 may be used in conjunction with introducer 1 to deploy electrode 50. Guidewire is inserted into the proximal port of handpiece 18 (not shown in FIG. 6) so that the distal portion 30 of the guidewire and plug 34 extend distally beyond tubular member 14 of introducer 1. The electrode assembly 50 is then mounted on plug 34 of the guidewire by inserting distal tip portion 30 of guidewire through the lumen of the electrode assembly 50. As discussed above, the diameter of the plug is large enough to form a press fit inside the inner diameter of the electrode coil 50. The length of plug 34 may be selected based on the length of the electrode assembly and the desired tightness of the fit between the electrode assembly and plug. If a looser fit is desired, the plug may be selected to be longer than if a tighter fit is desired so that the electrode assembly may be readily slipped off guidewire 20 during deployment.

According to the embodiment of the assembly shown in FIG. 6, the inner lumen of introducer 1 is large enough to accommodate both guidewire 20 and the lead body 54. In this embodiment, the lead body is loaded into the inner lumen of introducer 1 either before, or after, electrode assembly 50 has been coupled to plug 34 of guidewire 20. The entire assembly including introducer 1, guidewire 20, and electrode assembly 50 may then be introduced into a patient's vascular system in the manner known in the art. Steering of the assembly may be accomplished using deflection wire 23 to shape the distal tip portion 30 of the guidewire. As discussed above, this may be accomplished by applying a tension force to the proximal end of the deflection wire. Alternatively, in another embodiment of guidewire 20 not including tension wire 23, the distal tip portion may be manually shapeable. Rotation of the guidewire allows the distal tip to rotate so that the assembly may be maneuvered around the torturous curves of the vascular system. Because plug 34 is free to rotate, steering may be accomplished without the need to rotate the electrode assembly 50.

The electrode assembly of the current invention is particularly adapted for placement in the coronary sinus and the cardiac great vein, although the electrode may also be placed in other locations in the vasculature such as the middle cardiac vein. Navigation is aided by the use of the radiopaque tubular member 14. Once the proper implant site has been reached, the electrode may be displaced from plug 34 by advancing the introducer forward such that tubular member 14 engages the proximal portion of electrode assembly 50. Alternatively, the introducer may remain stationary while the guidewire 20 is pulled in a proximal direction. In either case, the applied force dislodges the electrode assembly at the desired implant site. The guidewire may then be withdrawn from introducer 1, and the introducer may be retracted over the lead body. In the embodiment shown in FIG. 6 wherein the lead body 54 passes through the inner lumen of introducer 1 defined by coil 8, a low profile lead connector is needed to remove the introducer. However, a splittable introducer of the type described above in reference to FIG. 2 may be employed in the alternative so that the lead may utilize a connector of any size.

FIG. 7 is a cutaway side view of another embodiment of the electrode assembly and the introducer. In this embodiment, electrode assembly 58 comprises one or more conductive rings 60 mounted in a tubular housing 62 and electrically connected to the lead body conductor by one or more jumper wires 61 provided in the walls of the tubular housing 62. The tubular housing may be formed of an insulative material such as a biocompatible polymer like silicone rubber. Alternatively, tubular housing 62 may be formed of a conductive material such that the entire housing serves as the electrode, making the conductive ring unnecessary. In either embodiment, the inner diameter of tubular housing 62 is sized to receive plug 34 in a press fit as is described above. The proximal end of electrode assembly 58 is coupled at one side to lead 64, leaving the inner lumen of the electrode assembly free to accept guidewire 20 as shown in FIG. 7.

FIG. 7 further illustrates introducer 63, which is an alternative embodiment of the introducer discussed above. In this embodiment, lead body 64 is not inserted into the inner lumen of the introducer. Instead, the lead body lies along the outer side of introducer 63. The lead is maintained in this position during implant by a grooved member 66 which is formed in the outer surface at the proximal end of introducer 63. If desired, grooved member 66 may be replaced by multiple grooved members spaced apart near the proximal end of the introducer. This embodiment of introducer 63 has the advantage of being smaller, since inner lumen 65 need only be sized large enough to accommodate plug 34, not lead 64.

Once the electrode assembly 58 has been delivered to the implant site the lead body 64 may be released from grooved member 66. Because the grooved member is located at the proximal end of introducer 63 at a location that remains outside the vascular system, lead body 64 may be easily removed from the grooved member in a manner that does not dislodge the electrode assembly from the implant site when the electrode is deployed.

It may be noted that the electrode assembly of FIG. 7 may be used with the introducer 1 shown in FIG. 6 for receiving lead body 64. Alternatively, the electrode assembly of FIG. 6 may be used with the introducer of FIG. 7 such that lead 54 lies outside of introducer 63.

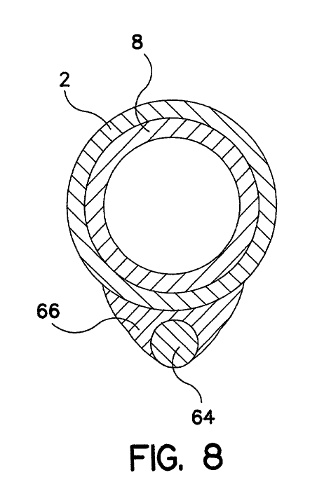

FIG. 8 is a cross-sectional view of the introducer at line 8-8, and illustrates lead body 64 retained in grooved member 66.

The guidewire discussed above utilizes plug 34 to engage the electrode assembly. Other embodiments of the guidewire may be used with any of the introducer and electrode assembly embodiments discussed above.

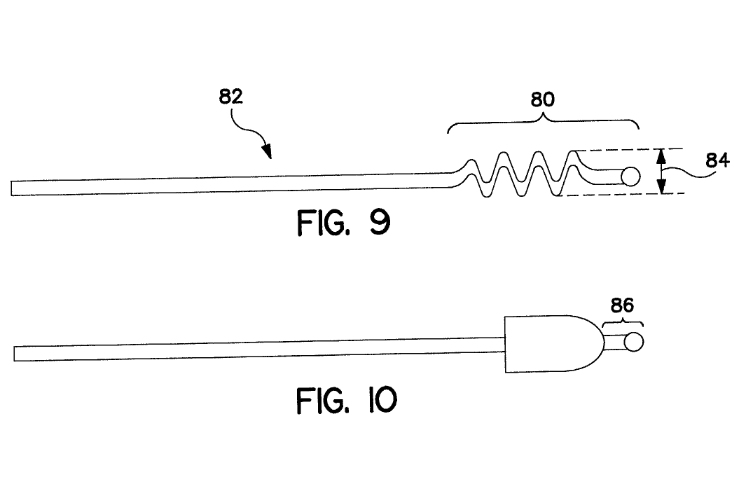

FIG. 9 is a side plan view showing an alternative embodiment of a guidewire that may be used in conjunction with the current invention. The guidewire of this embodiment may be of any of the constructions discussed above in reference to guidewire 20. In this embodiment, the bent distal tip region 80 of guidewire 82 includes multiple bends that define an outer diameter 84 that is sized to form a press fit with the electrode assembly 49 or 58.

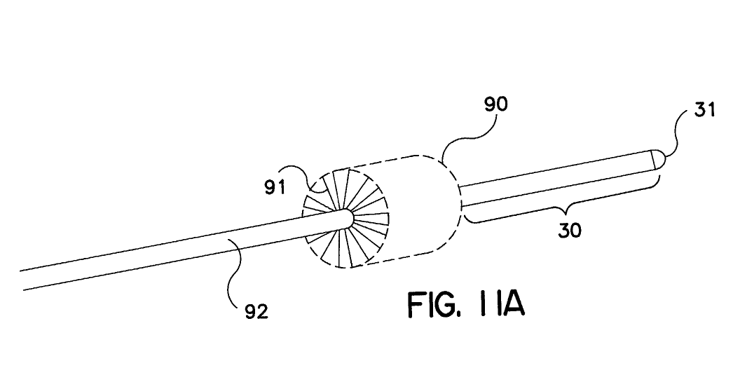

FIG. 10 is a side plan view showing an alternative embodiment of a guidewire that includes a shorter distal tip region 86 than is provided by previously-discussed guidewire designs.

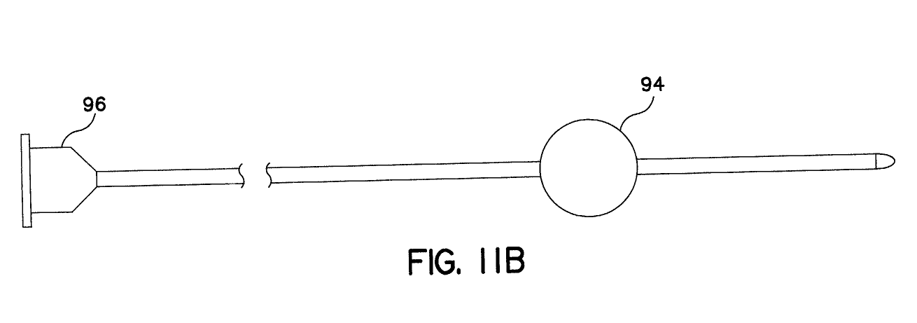

FIG. 11A is a perspective view of yet another embodiment of guidewire 20 including a brush member 90 (shown dashed) defined by soft deformable bristles 91 extending radially from the guidewire body 92 and adapted to form a press fit with the inner lumen of an electrode assembly. In this embodiment, the inner lumen of the introducer may be sized smaller than the outer diameter of the brush member since the bristles are capable of deforming when being advanced or retracted from the introducer. The guidewire body 92 may be formed according to any of the constructions discussed above. In yet another embodiment, deformable bristles may extend from a tubular core member that is mounted on grooved portion 36 (FIG. 3A) so that the brush member rotates independently of the guidewire in a manner similar to that discussed above in reference to plug 34.

FIG. 11B is yet another embodiment of the current invention in which the retention device is an inflatable member 94 such as a balloon provided on the body of the guidewire. This inflation member may be formed using any of the biocompatible materials known in the art for similar purposes. The guidewire includes a inner lumen in fluid communication within the inflation member to allow the inflation member to be inflated via an injection port 96 provided at the proximal end of the guidewire. The inflation member is expanded to retain the electrode in position on the guidewire during placement, and is then deflated to allow for electrode deployment. If it is necessary to reposition the electrode, the inflation member may be re-inflated, the guidewire moved, and the process repeated. This embodiment has the advantage of being useful with an introducer having an inner lumen sized only slightly larger than the body of the guidewire.

The above discussion describes the manner in which the current invention may be used to deploy an electrode coupled to a guidewire assembly. The introducer structure of the current invention may also be used to position an electrode over a previously-placed lead or electrode.

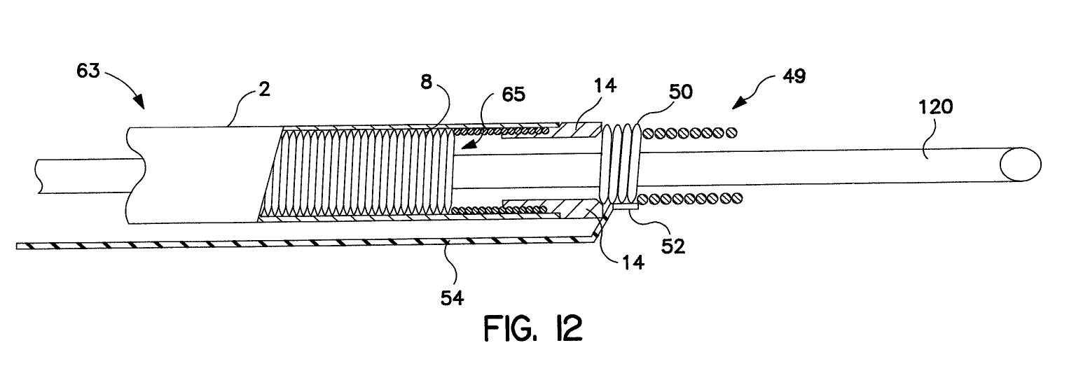

FIG. 12 is a cutaway side view illustrating an alternative embodiment of the introducer wherein the introducer does not include a member for engaging the inner diameter of an electrode assembly. According to one manner of use, this embodiment of the guidewire 120 is navigated to a predetermined implant site using one or more internal pull wires included within the guidewire body, or by employing a manually deflectable guidewire tip. Once the guidewire is positioned at the implant site, an electrode assembly shown as electrode assembly 49 is threaded onto the proximal end of the guidewire 120. The distal end of introducer 63 is then also threaded onto guidewire 120. The introducer is employed to push the electrode assembly 49 over the wire to the desired location. Guidewire and introducer 63 are thereafter removed.

The foregoing method may also be employed to deploy an additional electrode over a previously implanted lead. In this case, the procedure would be similar except that the first lead would remain implanted with the electrode assembly of the second lead positioned over the body of the first lead.

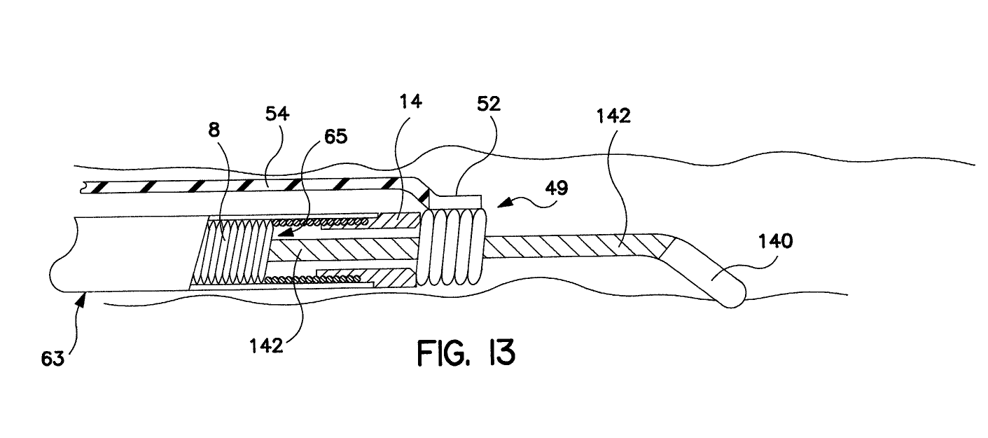

FIG. 13 is a cutaway side view illustrating a previously implanted lead body being used to implant additional electrodes. Previously implanted lead 142 may be connected to an implantable pulse generator in any manner known in the art. To perform the implant of an additional electrode assembly 49, the electrode assembly is threaded over the proximal end of lead body 142 via an extension of the lead body. To accomplish this, the lead body must be coupled to a low-profile connector. The extension of the lead may be made by inserting an elongated wire into the stylet lumen of the lead or by coupling a wire to the connector of the lead. Introducer 63 is also threaded over the lead body at the proximal end of the electrode assembly so that the introducer may push the electrode assembly over the lead 142 to a desired implant site. The introducer may then be withdrawn. The addition of electrodes in this manner may be desirable if a defibrillation electrode is to be added over a previously-placed pacing lead 142. Alternatively, if lead 142 carries a defibrillation electrode and it is determined that the defibrillation threshold is too high, an additional defibrillation electrode may be added to increase efficacy. In yet another situation, the addition of another pacing electrode can convert a unipolar electrode to a multipolar application. More than one electrode may be placed over lead 142 in this manner.

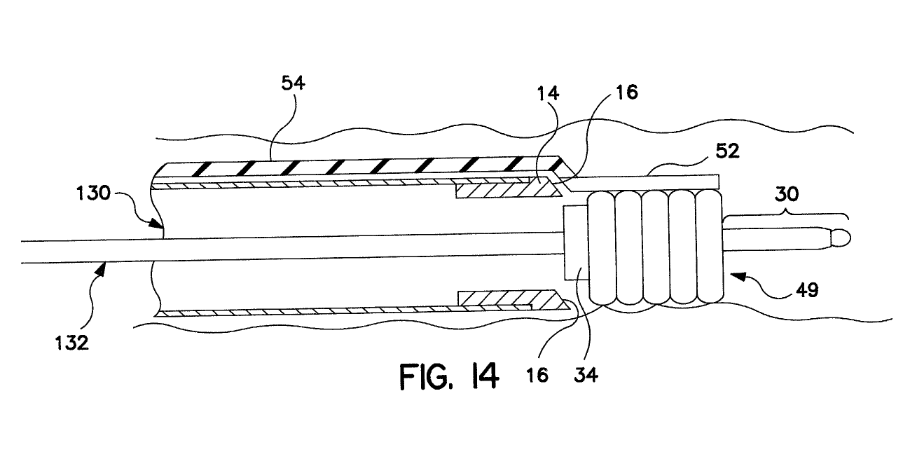

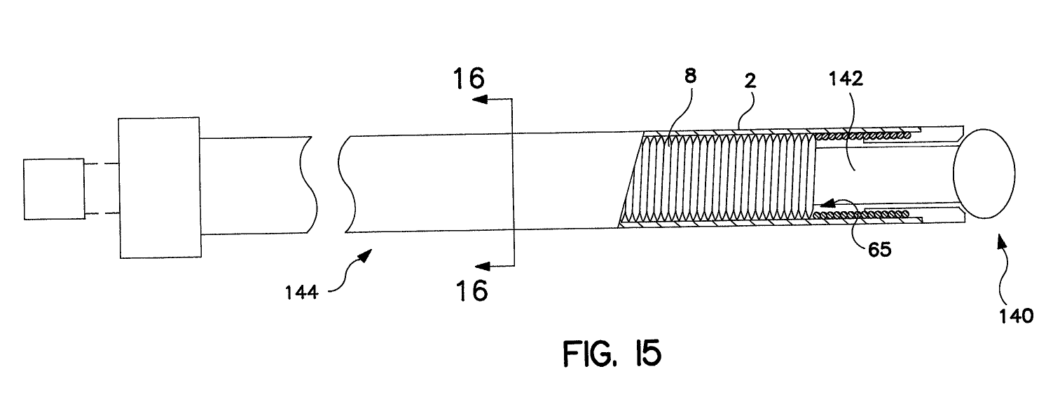

FIG. 14 illustrates the manner in which the current inventive system may be employed to reposition an electrode from a first implant site to another location. An introducer 130 that may take the form of any of the embodiments discussed above may be navigated to the initial implant site and aligned with the electrode assembly using a nesting taper 16 provided in fluoroscopic tubular member 14 of the introducer. A guidewire 132 having any of the embodiments discussed above is then inserted through the introducer. The electrode retention member shown for exemplary purposes as plug 34 (FIG. 3A) may then be inserted into the electrode assembly. This may be accomplished by applying pressure to the proximal end of lead 132 to maintain the proximal edge of the conductor against nesting taper 132 as forward pressure is applied to the proximal end of guidewire 132. When plug 34 is engaged with the electrode assembly, the electrode may be re-positioned by steering the guidewire and the introducer to a new implant site using distal tip 30.

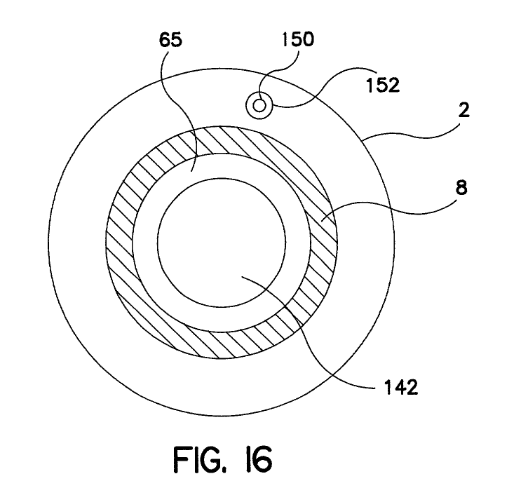

FIG. 15 is a side cutaway view illustrating the manner in which an introducer embodiment of the current invention may also be employed to deploy an electrode assembly 140 that does not have a lumen for the passage of a guidewire, and which is coupled to a lead body 142 that is axially aligned with the electrode. Because electrode assembly 140 and/or lead body 142 do not contain a lumen for a stiffening stylet, and because the lead body 142 is of insufficient stiffness to push the electrode assembly 142 forward into the vascular system for implant, introducer 144 having any of the embodiments discussed above may be used to steer the electrode assembly into place. In one embodiment, the introducer may include one or more pull-wires embedded in the side walls of the introducer and coupled to the distal end of the introducer. These one or more pull-wires extend the length of the introducer and are capable of deflecting the introducer distal tip 144 when tension is applied to the proximal end of the pull-wire. This deflection aids in steering the introducer and electrode to the site of implant.

FIG. 16 is a cross-sectional view of the introducer at line 16-16 of FIG. 15 illustrating a pull-wire 150 housed within a lumen 152 in the side wall of the introducer 144. Multiple ones of these pull-wires may be included at various locations around the periphery of the introducer 144. The application of tension on pull-wire 150 will result in deflection of the distal tip of introducer 144 to thereby aid in navigating the assembly within the vascular system.

Variations and modifications to the present invention may be possible given the above disclosure. However, all such variations and modifications are intended to be within the scope of the invention claimed by this letters patent.

In conjunction with the above disclosure, we claim:

What is claimed is:

1. A system for delivering an electrode assembly to an implant site, comprising:
a delivery device having an electrode retention member to couple to the electrode assembly; and
an introducer having an inner lumen sized to receive the delivery device, the introducer further having a distal tip adapted to mate with the electrode assembly when the electrode assembly is retained by the electrode retention member and the delivery device is located within the inner lumen, wherein the introducer is adapted to transfer the electrode assembly and the delivery device to the implant site.

2. The system of claim 1, wherein the delivery device includes a steerable distal tip.

3. The system of claim 2, wherein the steerable distal tip is coupled to a deflection wire extending to a proximal portion of the delivery device.

4. The system of claim 2, wherein the steerable distal tip is manually shapeable.

5. The system of claim 1, wherein the distal tip of the introducer is adapted to selectively disengage the electrode assembly from the electrode retention member.

6. The system of claim 1 wherein the electrode retention member is independently rotatable with respect to the delivery device.

7. The system of claim 6, wherein the delivery device includes a grooved portion, and wherein the electrode retention member is coupled to the grooved portion.

8. The system of claim 6, wherein the electrode retention member is a plug member capable of forming a press fit with the electrode assembly.

9. The system of claim 1, wherein the electrode retention member is a shaped distal tip portion of the delivery device capable of forming a press fit with the electrode assembly.

10. The system of claim 6, wherein the electrode retention member includes multiple radially-extending bristles capable of forming a press fit with the electrode assembly.

11. The system of claim 1, wherein the electrode retention member is an inflatable member.

12. The system of claim 1, wherein the introducer includes a sheath and a coil disposed within the sheath to prevent kinking of the introducer.

13. The system of claim 1, wherein the introducer is splittable.

14. The system of claim 1, wherein the introducer includes a proximal region, a distal region, and fluoroscopic member included within the distal region.

15. The system of claim 1, wherein the inner lumen is sized to receive a lead extending from the electrode assembly.

16. The system of claim 1, wherein the introducer includes a proximal section and a grooved member located at the proximal section, the grooved member adapted to engage a lead coupled to the electrode assembly.

17. A method of placing an electrode assembly within a living body, including the methods of:
a.) providing a delivery device having a proximal section, a distal section, and an electrode retention member located within the distal section;
b.) advancing the delivery device through an inner lumen of an introducer so that the electrode retention member extends beyond a distal end of the introducer;
c.) coupling the electrode assembly to the electrode retention member;
d.) applying a push force to the introducer to transfer the introducer, the delivery device, and the electrode assembly to a point of implant in the patient's body; and
e.) employing the introducer to dislodge the electrode assembly from the electrode retention member.

18. The method of claim 17, wherein the electrode assembly has been previously implanted at a first implant site within the patient's body, wherein method c.) is performed by coupling the electrode assembly to the electrode retention member at the first implant site, and wherein method d.) is performed to locate the electrode assembly at a second implant site.

19. The method of claim 17, wherein method c.) includes engaging the electrode assembly to the electrode retention member with a press fit.

20. The method of claim 17, wherein method d.) includes the method of steering the introducer, the delivery device, and the electrode assembly to the point of implant using the delivery device.

21. The method of claim 20, wherein the method of steering includes the method of deflecting a distal tip of the delivery device by applying tension to a pull-wire attached to the distal tip of the delivery device.

22. The method of claim 20, wherein the method of steering includes the method of rotating a preformed distal tip of the delivery device.

23. The method of claim 22, wherein the method of rotating including rotating the preformed distal tip of the delivery device independently of the electrode retention member.

24. The method of claim 17, wherein the coupling method c.) occurs outside of the patient's body.

25. The method of claim 17, wherein the coupling method c.) includes the method of mating a distal end of the introducer with the electrode assembly.

26. The method of claim 17, wherein the coupling method c.) further includes introducing a lead coupled to the electrode assembly through the inner lumen of the introducer.

27. The method of claim 17, wherein the coupling method c.) further includes attaching a lead coupled to the electrode assembly to a coupling member located on an exterior surface of the introducer.

28. The method of claim 17, and further including the method of splitting the introducer to remove the introducer from the patient's body.

29. The method of claim 17, wherein the electrode retention member is inflatable, and wherein the coupling method c.) includes inflating the electrode retention member to engage the electrode assembly.

30. A method of utilizing a delivery device having a distal end positioned within a body to deploy an electrode assembly within the body, including the methods of:
a.) coupling the electrode assembly to a proximal end of the delivery device;
b.) coupling an introducer to the proximal end of the delivery device at a position proximal to the electrode assembly;
c.) mating a distal end of the introducer with a proximal end of the electrode assembly;
d.) applying a push force to the introducer to transfer the distal end of the introducer and the electrode assembly to a predetermined location within the body; and
e.) withdrawing the introducer from the body.

31. The method of claim 30, wherein the delivery device is a previously-placed lead.

32. The method of claim 30, wherein method a.) includes the method of advancing the delivery device through a lumen of the electrode assembly.

33. The method of claim 30, wherein method c.) includes the method of mating a tapered member at the distal end of the introducer to the proximal end of the electrode assembly.

34. The method of claim 30, and further including the method of repeating methods a.) through d.) to deliver a second electrode assembly to a second predetermined location with the patient's body.

35. The method of claim 30 wherein method d.) includes the method of steering the introducer to the second predetermined location within the patient's body using at least one pull-wire.

36. The method of claim 30, wherein method e.) includes the method of splitting the introducer to remove the introducer from the delivery device.

37. The method of claim 30, wherein the electrode assembly includes at least one defibrillation electrode.

38. The method of claim 30, wherein the electrode assembly is for use in a multipolar pacing application.
